(19) United States
(12) Patent Application Publication
(10) Pub. No.: US 2025/0144289 A1
(43) Pub. Date: May 8, 2025

(54) MEDICAL DEVICE WITH ACOUSTIC AND/OR HAPTIC AND/OR OPTICAL FEEDBACK

(71) Applicant: B. Braun Melsungen AG, Melsungen (DE)

(72) Inventors: Hidayet Akkus, Kassel (DE); Michael Kauba, Fuldabrueck (DE); Johannes Diehl, Melsungen (DE); Markus Vogel, Katlenburg-Lindau (DE)

(21) Appl. No.: 18/938,762

(22) Filed: Nov. 6, 2024

(30) Foreign Application Priority Data
Nov. 7, 2023 (EP) 23 208 191.9

(51) Int. Cl. A61M 5/142

(52) U.S. Cl. CPC A61M 5/142; A61M 2205/505; A61M 2205/581; A61M 2205/582; A61M 2205/583

(57) ABSTRACT

A medical device includes an input unit configured to receive an input operation performed by a user for operating the medical device. A feedback unit is configured to output haptic feedback and/or acoustic feedback and/or optical feedback to the user in response to the input operation.

### CROSS-REFERENCE TO RELATED APPLICATION

This application claims priority under 35 U.S.C. § 119 to European Application No. 23 208 191.9, filed on Nov. 7, 2023, the content of which is incorporated by reference herein in its entirety.

### FIELD

The present disclosure relates to a medical device comprising an input unit for receiving an input operation performed by a user and a feedback unit for outputting a feedback in response to the input operation. The medical device may in particular be an infusion pump.

### BACKGROUND

Currently, medical devices almost exclusively comprise resistive touchscreens and/or mechanical/haptic buttons in order to receive an input operation performed by a user for operating the medical device. The input operation made via the resistive touchscreen or a mechanical button is detected via a redundant or even diverse detection system in order to ensure a high level of dependability/safety when receiving the input operation. This leads to an increased demand for components or software and, in the case of the diverse detection system, even to increased development costs. In addition, a resistive touchscreen only provides a limited resolution for defining areas for receiving different input operations, so that it is often necessary to switch between different displays on the resistive touchscreen.

### SUMMARY

It is therefore the object of the present disclosure to eliminate or at least reduce these disadvantages. In particular, it is the object of the present disclosure to provide a medical device which ensures a high level of safety when receiving an input operation for operating the medical device performed by a user.

The object is solved by a medical device as described herein. Advantageous embodiments are explained below.

A medical device according to the disclosure comprises an input unit configured to receive an input operation performed by a user for operating the medical device. Via the input operation, a treatment performed with the medical device can be controlled and/or parameterized, i.e. the treatment can be started or stopped and parameters related to the treatment can be set before or during the treatment. The medical device may in particular be an infusion pump. Alternatively, the medical device may be an extracorporeal blood treatment device, preferably a dialysis machine, or a control device for a medical or surgical tool, e.g. a drill, a milling machine, or a saw. A large number of other alternative embodiments of the medical device are conceivable.

The medical device also has a feedback unit configured to output or return feedback to the user in response to the input operation. The feedback may be a haptic or mechanical feedback, or an acoustic feedback, or a combination of haptic and acoustic feedback. In other words, the feedback may be a haptic or mechanical feedback and/or an acoustic feedback. Consequently, the feedback unit is capable of providing only acoustic feedback or only haptic/mechanical feedback. However, the feedback unit may also output a combination of acoustic and haptic/mechanical feedback. It is also conceivable that the feedback unit outputs optical feedback, for example in the form of a light signal, e.g. from an LED or a display on a touchscreen, in addition to or as an alternative to the acoustic and/or haptic feedback. A combination of optical feedback with acoustic feedback and/or haptic feedback is therefore also conceivable, as is purely optical feedback. Accordingly, the user of the medical device receives feedback from the feedback unit immediately after performing the input operation via the input unit. In this way, the receipt of the input operation is confirmed to the user of the medical device, so that safety is guaranteed when performing the input operation. In addition, it is no longer necessary to provide a redundant or even a diverse detection system for detecting the input operation.

Preferably, the input unit may be configured to receive different types of input operations. Accordingly, a separate type is settable for each input operation. The type may be defined according to safety criteria for the treatment performed with the medical device. The feedback unit may then be configured in such a way that it outputs different types of feedback in response to the different types of input operations. Consequently, not only the receipt of the input operation, but also the type of input operation is returned or confirmed to the user by the feedback unit, so that safety is increased when performing the input operation.

Advantageously, the feedback unit may be configured such that an intensity or frequency of the haptic feedback and/or a sound level of the acoustic feedback and/or a brightness of the optical feedback is settable. Accordingly, the user of the medical device can set the intensity/frequency or the sound level according to his/her preferences in order to reliably perceive the feedback that is output in response to the received input operation. The same applies to setting the brightness of the optical feedback. For example, setting an increased intensity or an increased frequency is advantageous in a case where the user is wearing gloves during the input operation. This can further increase safety when performing the input operation.

Advantageously, the types of input operations may be grouped into a first group. The feedback unit may then be configured to output a predetermined and non-changeable feedback in response to a type of input operation of the first group. Types of input operations that are common to a large number of different medical devices may be grouped into the first group. In addition, types of input operations that are highly relevant for the reliability or safety of a treatment performed with the medical device may be grouped into the first group. Examples of this are a home button, an on/off button, a stop button, or a start button. When receiving such an input operation, the same feedback is always returned so that the user can immediately validate the execution of this particular input operation. For example, when the stop button is pressed, the same combination of haptic feedback and acoustic feedback is always output. Of course, optical feedback may also be provided. Since this type of feedback cannot be changed in response to the operation of the stop button, the user can validate the operation of the stop button without much thought even when operating a different medical device. Consequently, a standard feedback can be established for the medical devices of a company or even for medical devices of a certain type, e.g. for infusion pumps. In this way, safety in the operation of medical devices can be further increased.

In a further development, the feedback unit may be configured in such a way that it outputs an, in particular in each case, dedicated feedback in response to an input operation with an incorrect value, an invalid value, or an incorrect input. This immediately alerts the user to a critical input operation and an input can be corrected immediately.

Furthermore, the types of input operations may be grouped into a second group. The feedback unit may then be configured such that a feedback output in response to a type of input operation of the second group is settable. In the second group, types of input operations may be grouped that are very specific and/or have little or no influence on the reliability or safety of a treatment performed with the medical device. Examples of this are a selection operation for selecting a specific menu item or switching between different displays. The feedback may be individually either haptic or acoustic feedback. The combination of haptic and acoustic feedback may also be defined individually. Optical feedback or a combination with optical feedback is also conceivable. Of course, feedback for the second group may also be switched off completely. Accordingly, a user may define the feedback that is output in response to the types of input operations of the second group entirely according to his/her individual preferences. This individual definition increases familiarity when operating the medical device and thus security.

The input unit may preferably have a touchscreen. This means that frequent switching between displays can be avoided and operation of the medical device is simplified. If the touchscreen is used exclusively as an input unit, a clearly arranged appearance of the medical device can also be achieved. In addition, cleaning or disinfection of a medical device that has a touchscreen is simplified or improved compared to a medical device that has a plurality of mechanical buttons that form edges and grooves on the outer surface of the medical device. Due to the output of feedback in response to an input operation, the touchscreen also offers the advantage of a button that provides haptic perception during an input operation. This is also achieved in a similar way by acoustic feedback. The touchscreen also offers the further advantage that it may also provide optical feedback.

Advantageously, the touchscreen may have an ESD glass front, and/or toughened safety glass front, or a chemically hardened glass front. These glass fronts offer advantages in terms of robustness, biocompatibility and cleaning/cleaning agents. Accordingly, a reliable and durable medical device can be achieved.

The input unit may have at least one button. The button may preferably be a capacitive button. The button is used in particular for an input operation that can be carried out quickly by a user without a long search in menus. This includes, for example, switching the medical device on/off or stopping a treatment performed with the medical device. By providing the button, safety when using the medical device can be improved.

Preferably, the feedback unit may have a vibrating element. The vibrating element may in particular be a Linear Resonant Actuator (LRA). In this way, a fast and intensive feedback can be given in response to the input operation. Alternatively, the vibrating element may be a vibration motor or a piezo actuator. Piezo actuators require a small installation space and provide a short response time.

Preferably, the vibrating element may be implemented using an integrated circuit. In this way, the vibrating element can be easily integrated into the medical device.

Furthermore, the integrated circuit may be drivable via a bus, in particular I²C (Inter-Integrated Circuit) bus, SPI (Serial Peripheral Interface) bus, One Wire bus, UART (Universal Asynchronous Receiver Transmitter) bus, CAN (Controller Area Network) bus, Bit-Bang (e.g. GPIO (General Purpose Input/Output) bus). This makes it easy to integrate the vibrating element into the medical device.

In addition, the medical device may already comprise a loudspeaker and/or a light source, in particular an LED, and/or a touchscreen. In this case, the feedback unit can be realized by software configured to tap the input operation and to output a signal for outputting the acoustic feedback to the loudspeaker and/or a signal for outputting the optical feedback to the light source and/or to the touchscreen in response to the tapped input operation. Accordingly, the feedback unit can be easily retrofitted in an existing medical device that already has a loudspeaker and/or a light source and/or a touchscreen, e.g. via a firmware update.

### BRIEF DESCRIPTION OF THE DRAWINGS

In the following, the medical device according to the present disclosure is described with reference to the attached Figures, in which identical elements are provided with the same reference signs. In the attached Figures:

FIG. 1 shows a schematic view of a medical device that receives an input operation from a user and outputs feedback in response to the input operation;

FIG. 2 shows a schematic block diagram of the medical device; and

FIG. 3 shows a sequence diagram for a medical device in which the feedback unit for outputting an acoustic feedback is implemented by software.

### DETAILED DESCRIPTION

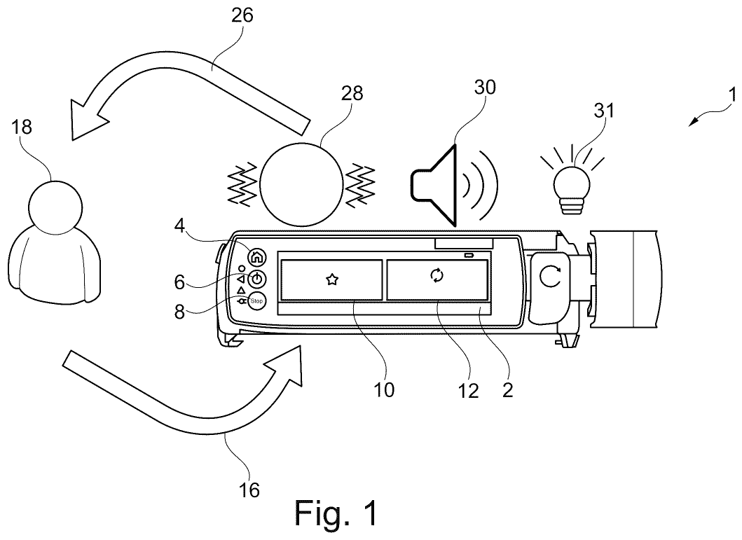

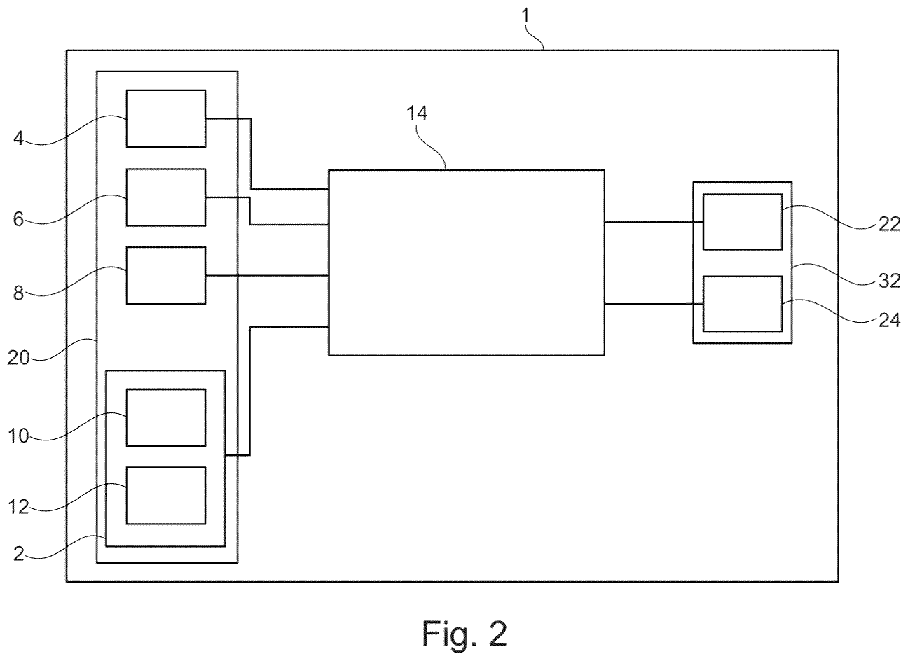

FIG. 1 shows a medical device 1 according to the disclosure, which in the present embodiment is configured as an infusion pump. FIG. 2 furthermore shows a schematic block diagram of the medical device 1. The medical device 1 comprises a touchscreen 2 and buttons 4, 6, and 8.

The touchscreen 2 is configured to receive touching from a finger or another input device, such as a pen, in order to trigger corresponding processing in the medical device 1. A first input symbol 10 and a second input symbol 12 are displayed on the touchscreen 2. The input symbols 10, 12 are displayed on the touchscreen 2 by executing a software by a microcomputer 14 of the medical device 1 and when touching the area of the input symbols 10, 12 on the touchscreen 2, a corresponding processing is executed by the microcomputer 14. In the present embodiment, the first input symbol 10 is used for selecting or setting a profile of a new patient and the second input symbol 12 is used for selecting the profile of the same patient.

In the medical device 1 shown in FIG. 1, the buttons 4, 6, 8 are arranged next to the touchscreen 2 and one below the other. In the present case, the buttons 4, 6, 8 are configured as a home button 4, an on/off button 6, and a stop button 8. The buttons 4, 6, 8 offer the advantage that they can be operated quickly without a long search in a menu structure displayed on the touchscreen 2. The buttons 4, 6, 8 may be arranged behind a foil or a glass surface. This avoids edges or grooves around the buttons 4, 6, 8 in which germs can accumulate and which cannot be reliably cleaned or disinfected.

The touchscreen 2 and the buttons 4, 6, 8 are configured to receive an input operation 16 performed by a user 18 of the medical device 1 and to forward it to the microcomputer 14 so that corresponding processing is initiated in the microcomputer 14. As a result of the processing, the microcomputer 14 can output signals in order to control or parameterize a treatment carried out with the medical device 1. The touchscreen 2 and the buttons 4, 6, 8 thus together correspond to an input unit 20 according to the preferred embodiment, as shown in FIG. 2. However, it is also conceivable that the medical device 1 has only the touchscreen 2 or only the buttons 4, 6, 8, possibly together with other buttons as the input unit 20.

The medical device 1 according to the disclosure further comprises, as shown in FIG. 2, a vibrating element 22 and a loudspeaker 24. Upon receipt of the input operation 14 via the input unit 20, feedback 26 is output to the user 18 according to the present disclosure, as shown in FIG. 1. As shown in FIG. 2, the output of the feedback 26 is also triggered by the microcomputer 14 upon receipt of the input operation 14. The feedback 26 may be a haptic feedback 28 by the vibrating element 22 or an acoustic feedback 30 by the loudspeaker 24. In addition, the feedback 26 may also be a combination of acoustic and haptic feedback 28, 30. The vibrating element 22 and the loudspeaker 24 thus together correspond to a feedback unit 32, as shown in FIG. 2. It is also conceivable that the feedback unit 32 is formed only by the vibrating element 22 or only by the loudspeaker 24. It is also conceivable that the feedback 26 is an optical feedback 31, for example in the form of a light signal displayed on the touchscreen 2 or a display on the touchscreen 2, in addition to or as an alternative to the acoustic and/or haptic feedback 28, 30. In this case, the feedback unit 32 is also formed by the touchscreen 2. Alternatively, an additional light source, such as an LED, may be provided for outputting the optical feedback 31. The light source is then also part of the feedback unit 32.

The vibrating element 22 is configured as a Linear Resonant Actuator (LRA). Alternatively, the vibrating element 22 may also be formed by a vibration motor or a piezo actuator. It has proven to be preferable if the vibrating element 22 is implemented by an integrated circuit which is drivable by the microcomputer 14 via a bus, such as in particular an I²C (Inter-Integrated Circuit) bus, an SPI (Serial Peripheral Interface) bus, a One Wire bus, a UART (Universal Asynchronous Receiver Transmitter) bus, a CAN (Controller Area Network) bus, Bit-Bang (e.g. a GPIO (General Purpose Input/Output) bus).

As already mentioned, the medical device 1 comprises the loudspeaker 24. The loudspeaker 24 may also be provided on an integrated circuit. Advantageously, the loudspeaker 24 may be provided on the same integrated circuit as the vibrating element 22.

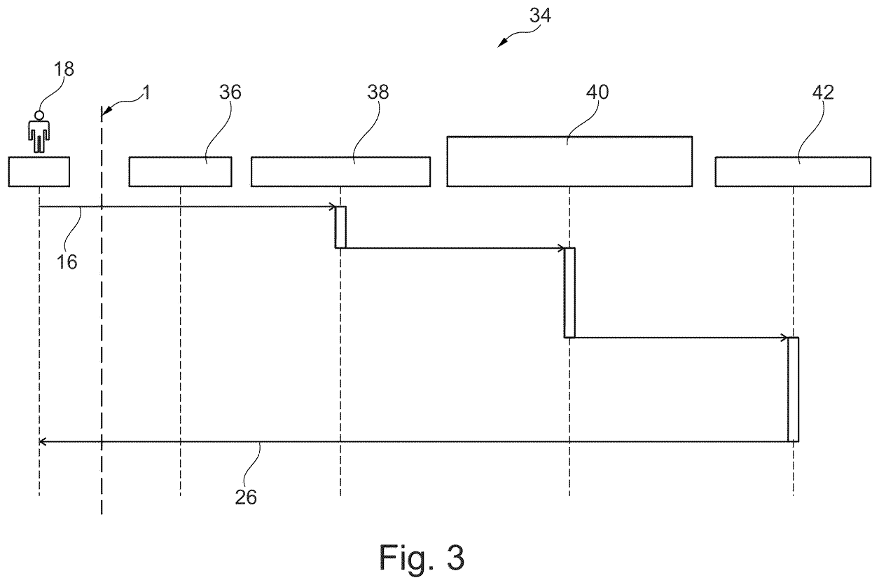

However, many medical devices 1 already have a loudspeaker 24 for outputting warning signals, so that it is advantageous to realize the feedback unit 32 for outputting the acoustic feedback 30 by software 34. A sequence diagram of the software 34 is shown in FIG. 3.

As shown in FIG. 3, a user 18 performs an input operation 16 on the touchscreen 2, on which input symbols 10, 12 are displayed via a graphics layer 36. A machine user interface controller (MUIC) 38 arranged downstream then detects touching on the touchscreen 2 in the area of the input symbols 10, 12. An event handler 40 picks up the detection of the input operation 16 by the MUIC 38 and causes an audio control 42 to output acoustic feedback 30 via the loudspeaker 24. Accordingly, the feedback unit 32 for outputting an acoustic feedback 30 can be subsequently implemented in a medical device 1 that already has a loudspeaker 24 in a simple manner, e.g. by a firmware update of the microcomputer 14. Similarly, the medical device 1 may already have a light source, e.g. an LED, or the touchscreen 2 and a lighting control may be implemented in addition or as an alternative to the audio control 42, so that optical feedback 31 is made possible.

As described above, different types of input operations 16 are entered into the input unit 20. The feedback unit 32 is configured in such a way that it outputs different types of feedback 26 in response to the different types of input operations 16. In the example described, a treatment can be interrupted by pressing the stop button 8. This represents a major intervention in the treatment being carried out with the medical device 1. On the other hand, the operation of the input symbol 10 to create a profile for a new patient represents processing that takes place before the treatment is carried out and therefore has hardly any influence on the treatment. Consequently, a different type of feedback 26 should preferably be output for the input operation 16 carried out by the stop button 8 than for the input operation 16 carried out by the input symbol 10. For example, a combination of haptic and acoustic feedback 28, 30 may be used for the operation of the stop button 8, so that the feedback can be detected by two different sensory organs. With the additional output of optical feedback 31, e.g. via the touchscreen, feedback can even be perceived by three sensory organs. In the case of operation of the input symbol 10, on the other hand, returning haptic feedback 28 may be sufficient, for example.

In addition, the feedback unit 28 may be configured such that an intensity or frequency of the haptic feedback 28 can be determined by the user 18. Similarly, a sound level of the acoustic feedback 30 may be set by the user 18. In this way, the user can adjust the feedback 26 according to his/her preferences or according to the environmental conditions. In a noisy environment, the sound level can be increased so that the acoustic feedback 30 is audible. In quiet environments, such as an operating theater, the sound level of the acoustic feedback 30 can be reduced and, in return, the intensity or frequency of the haptic feedback 28 can be increased so that the feedback 26 can be reliably detected. In addition, the output of optical feedback 31 can be advantageous in a quiet environment.

In addition, the types of input operations 26 may be grouped into a first group. Preferably, types of input operations 16 that are present in a large number of different medical devices 1 may be grouped into the first group. In addition, types of input operations 16 that are highly relevant for the reliability or safety of a treatment performed with the medical device 1 may preferably be grouped into the first group. The feedback unit 32 may then be configured to output a predetermined and non-changeable feedback 26 in response to a type of input operation 16 of the first group. For example, a predetermined combination of haptic and acoustic feedback 28, 30 may be defined for the stop button 8. This combination cannot be changed by a user 18. Accordingly, this feedback 26 is always output for different medical devices 1 when the stop button 8 is pressed, so that the user 18 is familiar with the feedback 26 and can reliably validate an operation of the stop button 8.

The feedback unit 20 may be configured in such a way that it outputs dedicated feedback 26 in response to an input operation 16 with an incorrect value, an invalid value, or an incorrect input. This immediately alerts the user to a critical input operation 16 and an input can be corrected immediately. For example, setting of a dosage that is too high or too low can be recognized reliably and immediately.

The types of input operations 16 may also be grouped into a second group. In the second group, types of input operations 16 may be grouped which are very specific and have little or no influence on the reliability or safety of a treatment performed with the medical device 1. The feedback unit 28 may then be configured such that a feedback 26 output in response to a type of input operation 16 of the second group can be determined. In the present example, input operations 16 performed via the input symbols 10 and 12 may be grouped in the second group, since their operation is performed before the patient is actually treated. The user 18 can customize the feedback 26 that is issued in response to the operation of the input symbols 10, 12 according to his/her preferences, so that familiarity with the medical device 1 is increased. For example, the feedback 26 may be set as a haptic feedback 28 to avoid disturbing noises. Alternatively, the output of feedback 26 may be switched off completely.

The medical device 1 thus makes it possible to increase safety when operating the medical device 1 by outputting feedback 26 in response to the input operation 16. By outputting standard feedback in response to certain input operations, which are often present in medical devices 1 and/or have a major influence on a treatment carried out with the medical device 1, the user 18 can safely record or validate the input operation 16 performed. In addition, the adjustability of the feedback 26, which is output in response to a less relevant input operation 16, increases the familiarity of the user 18 with the medical device 1, which also increases safety.

In the present disclosure, the medical device 1 has been described as an infusion pump. The present disclosure is not limited thereto and can also be applied to other medical devices 1 in which input operations can be performed.

### LIST OF REFERENCE SIGNS

1 medical device
2 touchscreen
4 home button
6 on/off button
8 stop button
10 first input symbol
12 second input symbol
14 microcomputer
16 input operation
18 user
20 input unit
22 vibrating element
24 loudspeaker
26 feedback
28 haptic feedback
30 acoustic feedback
31 optical feedback
32 feedback unit
34 software
36 graphics layer
38 Machine User Interface Controller (MUIC)
40 event handler
42 audio control

1. A medical device comprising:
an input unit configured to receive an input operation performed by a user for operating the medical device; and
a feedback unit configured to output haptic feedback, and/or acoustic feedback, and/or optical feedback to the user in response to the input operation.

2. The medical device according to claim 1, wherein:
the input unit is configured to receive different types of input operations; and
the feedback unit is configured to output different types of feedback in response to the different types of input operations.

3. The medical device according to claim 2, wherein:
the different types of input operations are grouped into a first group having a first type of input operation; and
the feedback unit is configured to output a predetermined and non-changeable feedback in response to the first type of input operation of the first group.

4. The medical device according to claim 3, wherein:
the different types of input operations are grouped into a second group having a second type of input operation; and
the feedback unit is configured such that a feedback output in response to the second type of input operation of the second group is settable.

5. The medical device according to claim 1, wherein the feedback unit is configured such that an intensity or frequency of the haptic feedback and/or a sound level of the acoustic feedback and/or brightness of the optical feedback is settable.

6. The medical device according to claim 1, wherein the feedback unit is configured to output a dedicated feedback in response to an input operation with an incorrect value, an invalid value, or an incorrect input.

7. The medical device according to claim 1, wherein the input unit has at least one button.

8. The medical device according to claim 1, wherein the feedback unit has a vibrating element.

9. The medical device according to claim 8, wherein the vibrating element is implemented using an integrated circuit.

10. The medical device according to claim 9, wherein the integrated circuit is drivable via a bus.

11. The medical device according to claim 1, wherein:
the medical device comprises a loudspeaker, and/or a light source, and/or a touchscreen, and
the feedback unit is realized by software configured to tap the input operation and to output a signal for outputting the acoustic feedback to the loudspeaker and/or a signal for outputting the optical feedback to the light source and/or to the touchscreen in response to the input operation.
